(19) United States
(12) Patent Application Publication
(10) Pub. No.: US 2025/0092073 A1
(43) Pub. Date: Mar. 20, 2025

(54) PHOSPHONIUM-BEARING CONJUGATED POLYELECTROLYTES

(71) Applicant: Board of Regents, The University of Texas System, Austin, TX (US)

(72) Inventors: Han Sun, Houston, TX (US); Isai Barboza-Ramos, San Antonio, TX (US); Xiaodan Wang, San Antonio, TX (US); Kirk S. Schanze, San Antonio, TX (US)

(21) Appl. No.: 18/811,575

(22) Filed: Aug. 21, 2024

Related U.S. Application Data
(60) Provisional application No. 63/533,901, filed on Aug. 21, 2023.

(51) Int. Cl. C07F 9/54; A61K 31/66; A61K 41/00; A61P 31/14

(52) U.S. Cl. CPC C07F 9/5449; A61K 31/66; A61K 41/0057; A61P 31/14

(57) ABSTRACT

A solution is described for therapeutic materials against antibiotic-resistant bacterial strains. A series of four PPE-based water-soluble conjugated polyelectrolytes containing phosphonium side chains of varying amphiphilic balance. The study of the photophysical properties revealed the efficient formation of 102 and ROS for their use as photosensitizers for light-activated antibacterial applications. A photosensitizer absorbs light to produce reactive oxygen species (ROS), and a photodynamic therapy (PDT) is widely used, in which the photosensitizer is excited upon irradiation with light of a specific wavelength from the outside to generate active oxygen species or free radicals, thereby inducing apoptosis of various lesions or cancer cells and destroying them.

### RELATED APPLICATION

The application is a US patent application claiming priority to U.S. Provisional Patent Application Ser. No. 63/533,901 filed Aug. 21, 2023 which is incorporated herein by reference in its entirety.

### STATEMENT REGARDING FEDERALLY FUNDED RESEARCH

None.

### FIELD OF THE INVENTION

Embodiments of the invention are directed to the field of microbiology and medicine.

### BACKGROUND

The widespread phenomenon of emerging antibacterial resistance has compromised typical therapeutic methods for overcome lethal pathogenic infections over the past decades.^{1,2} As stated in recent World Health Organization reports, the treatment with last-resort antibiotics continues to grow as frontline antibacterial drugs fail to relieve patients.³ Staphylococcus aureus and Escherichia coli are bacterial strains that consistently pose a threat to public health. Multidrug-resistant E. coli, for instance, is responsible for healthcare-associated infections in hospitalized patients of all ages that include the occurrence of septicemia, pneumonia cases, and urinary tract infections. Ampicillin, co-trimozazole, fluoroquinolones, and extended spectrum cephalosporines are among the antibiotics that have been shown inefficacy for E. coli resistant isolates.⁴ Similarly, S. aureus strains with notorious resistance to antibiotics remain a source of concern for causing complications to predisposed patients with acquired immunodeficiency syndrome and diabetes. Specially, invasive methicillin-resistant S. aureus occurrence remains high, implicating treatment for medical device management and procedure-associated infections.⁵ As the global crisis on antibiotic resistance progresses, noteworthy efforts focus on developing therapeutic materials against antibiotic-resistant bacterial strains.

Conjugated polymeric systems have drawn attention for their ability to efficiently inactivate harmful pathogenic strains, becoming an alternative for reducing the burden of infectious diseases.⁶⁻⁸ In particular, water-soluble conjugated polyelectrolytes (CPEs) have gained recognition as effective photosensitizers in phototherapeutics for targeting pathogenic bacteria. Conjugated polyelectrolytes are water-soluble polymers that possess π-electron delocalized backbones that enable exceptional light-harvesting properties and solubilizing pendant groups that improve the processability in aqueous conditions and biocompatibility.⁹ These properties lead to their usage in a wide range of applications, particularly in the fields of chemosensing and biosensing, optoelectronic devices as well as phototherapeutics.^{10,11} When used as photosensitizing agents, water-soluble CPEs can trigger a chain of chemical and biochemical reactions that render the target microorganisms inactive.¹²⁻¹⁴ Two key contributing mechanisms contribute to the overall inactivation process of pathogens. On the one hand, the cationic side-chains interact electrostatically with the bacterial membranes, causing cationic stress that ruptures the cell.¹⁵⁻¹⁷ On the other hand, when exposed to visible light, the polymer chains can photosensitize surrounding molecular oxygen molecules to produce reactive oxygen species (ROS), including singlet oxygen, hydroxyl radical, superoxide radical, and hydrogen peroxide.¹⁸⁻²⁰ By interacting and reacting with the cell membrane or organelles, the ROS damage and impair vital cell functions which cause synergistic photodynamic inactivation.

To date, various charged solubilizing functional groups have been incorporated to the design of biocidal CPEs, including quaternary ammonium, imidazolium, bis-imidazolium, and guanidinium functionality in the side chains or main chain. A relevant example includes the efficient light-activated antibacterial activity of PIM-2 and PIM-4.²¹ These polymers are cationic poly(phenylene ethylene)-(PPE-) based CPEs bearing imidazolium side chains that exhibit significant light-induced antibacterial activity at low concentration of 5 μg mL⁻¹ against gram-negative E. coli and Gram-positive S. aureus. In a further study, PPE-based conjugated polyelectrolytes with variable chains containing imidazolium solubilizing groups demonstrated to efficiently inactivate E. coli and S. epidermidis under light irradiation. The bacterial viability data suggest that the polymers with shorter main-chain lengths demonstrate greater bacteria-killing effects due to the high triplet excite state yield that produces a higher singlet oxygen quantum yield.²² Parallelly, the side-chain length of polyelectrolytes has been found to influence the aggregation state of the polymer, and therefore, the biocide efficacy when used as antibacterial agents.²³

The accessible tunability and versatility of CPEs have boosted the exploration of novel cationic pendant groups including the phosphonium functionality. An earlier study by Hladysh and coworkers described the synthesis and optical properties of a series of phosphonium-containing polythiophenes.^{24,25} Hu et al reported the design of thermally stable and multifunctional cationic phosphonium-based conjugated polyelectrolytes as a component of efficient polymer solar cells.²⁶ In addition, Zhang and colleagues studied regioregular polythiophenes with trimethyl-phosphonium moieties for imaging live cells.²⁷ Most recently, Zhou and coworkers reported the design of degradable pseudo conjugated polyelectrolytes nanoparticle with phosphonium sidechains that exhibited in vivo near-infrared-II photothermal antibacterial effect against E. coli and S. aureus bacterial strains.²⁸ In relation to biocidal activity, phosphonium moieties have been first incorporated into non-conjugated polymeric systems, demonstrating outstanding targeted activity against various pathogenic bacteria.²⁹⁻³¹ However, the study of PPE-based CPEs containing phosphonium pendant groups as photo-therapeutic agents remains limited, leaving room to investigate this class of materials against relevant bacterial strains.

There remains a need for additional CPE molecules for the treatment of bacterial infections.

### SUMMARY

The inventors have developed a solution for therapeutic materials against antibiotic-resistant bacterial strains. A series of four PPE-based water-soluble conjugated polyelectrolytes containing phosphonium side chains of varying amphiphilic balance. The study of the photophysical properties revealed the efficient formation of 102 and ROS for their use as photosensitizers for light-activated antibacterial applications. A photosensitizer absorbs light to produce reactive oxygen species (ROS), and a photodynamic therapy (PDT) is widely used, in which the photosensitizer is excited upon irradiation with light of a specific wavelength from the outside to generate active oxygen species or free radicals, thereby inducing apoptosis of various lesions or cancer cells and destroying them. The evaluation biocidal activity of these new cationic PPE-based CPEs represents an opportunity for targeting drug-resistant pathogens. Described herein is a dark and light-induced antimicrobial activity of PPh-2-3C, PPh-2-6C, PPh-4-3C, and PPh-4-6C (see the structures in FIG. 1) as novel PPE-based CPEs bearing phosphonium functional groups with different side-chain lengths. In certain embodiments the carbon sidechain can be 3, 4, 5, 6, 7, 8, 9, 10, 11, 12, 13, 14, 15 or more carbons. The ability of these phosphonium-functionalized CPEs to sensitize singlet oxygen under light exposure made them prospective candidates for further biocidal studies against E. coli and S. aureus bacteria. The light-activated biocidal action of the polymers is described at different concentrations, along with a quantitative assessment of the log reduction level of antibacterial activity utilizing the serial dilution method. In addition, fluorescence confocal imaging provided insights into the mechanism of antibacterial action. The outcomes also demonstrate the significance of taking amphiphilic balance and side chain length as important elements when employing water-soluble CPEs for antibacterial phototherapy.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1. Chemical structure of phosphonium-containing conjugated polyelectrolytes utilized for the antibacterial studies.

FIG. 2A-2D. Biocidal activity of phosphonium-containing CPEs toward E. coli in the (a) dark and (b) under light irradiation. Biocidal activity of phosphonium-containing CPEs toward S. aureus in the (c) dark and (d) under light irradiation. The light exposure was carried out using a 400 nm LED light with a power density of 16 mW/cm². CFU is the acronym for Cell Colony Forming Units. Values are expressed as means±SD (n=3, P<0.01).

FIG. 3A-3B. (a) Log reduction of CFUs determined by plate counting for 15 min irradiation with the phosphonium-containing CPEs at 20 μM against E. coli. (b) Log reduction of CFU determined by plate counting for 15 min irradiation with the phosphonium-containing CPEs at 10 μM against S. aureus. (The light irradiance was 16 mW/cm² for all cases).

FIG. 4. Confocal microscope images of E. coli incubated with PPh-2-3C, PPh-2-6C, PPh-4-3C, and PPh-4-6C at different incubation time and concentrations. Scale bar: 10 μm. The fluorescence lifetime is shown in a continuous pseudo-color scale (right) ranging from 0 to 5 ns.

FIG. 5. Confocal microscope images of E. coli treated with PPh-2-3C, PPh-2-6C, PPh-4-3C, and PPh-4-6C for 30 min at the concentration of 20 μM. Below is the corresponding plot intensity analyzed by Image J. The fluorescence lifetime is shown in a continuous pseudo-color scale (right) ranging from 0 to 3 ns.

FIG. 6. Confocal microscope images of S. aureus treated with PPh-2-3C, PPh-2-6C, PPh-4-3C, and PPh-4-6C for 15 min at the concentration of 5 μM. Below is the corresponding plot intensity analyzed by the Image J software. The fluorescence lifetime is shown in a continuous pseudo-color scale (right) ranging from 0 to 3 ns.

FIG. 7. Water-soluble CPs are multifunctional materials that incorporate charged pendants groups to overcome limitations on solubility and processability.

FIG. 8. Absorption and emission. PPh-2-6C and PPh-4-6C display a red shift in the absorption spectra. The extended alkyl side chains enable greater intramolecular separation of the cationic groups, therefore, the planarity and molecular orbital overlapping of the conjugated backbone is affected to a lesser extent compared to PPh-2-3C and PPh-4-3C.

FIG. 9. Transient absorption spectroscopy. Pump-probe transient absorption experiments proved the presence of a long-lived component attributed to the triplet excited state formed by intersystem crossing.

FIG. 10. ¹O₂ emission and reactive oxygen species. The steady-state near-infrared luminescent at 1270 nm confirmed the photosensitized production of singlet oxygen. PPh-2-3C and PPh-4-3C with shorter side-chains showed greater ROS generation due to the efficient 102 diffusion to react with the ADMA probe before decaying.

FIG. 11A-11D. Antibacterial activity. Results suggest the biocidal activity is higher after light irradiation (λ=400 nm, 15 min, 16 mW/cm²). The greater antibacterial efficiency of PPh-2-3C and PPh-2-6C could be attributed to the enhanced amphiphilic balance. Oxidative stress induces the degradation of the bacterial membrane and intracellular components. (A, B) E. coli viability before and after irradiation, respectively; (C, D) S. aureus viability before and after irradiation, respectively.

FIG. 12. Log reduction of colony forming units (CFUs).

FIG. 13. Confocal laser scanning microscopy.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

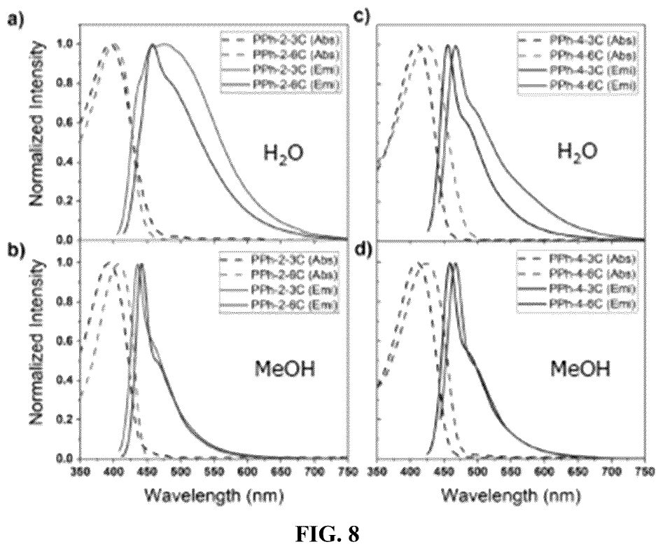

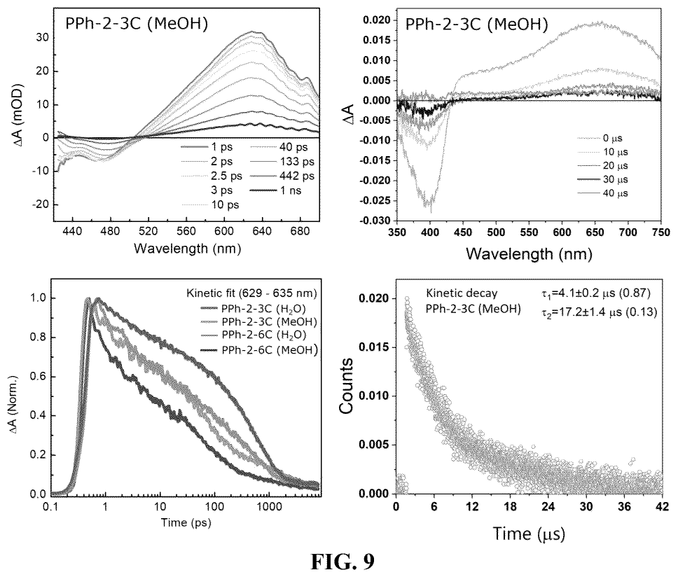

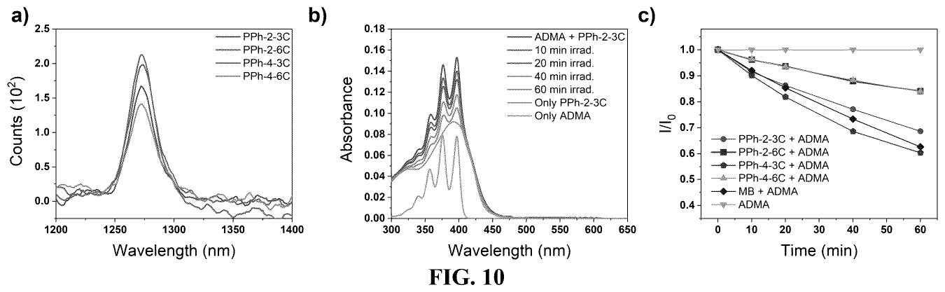

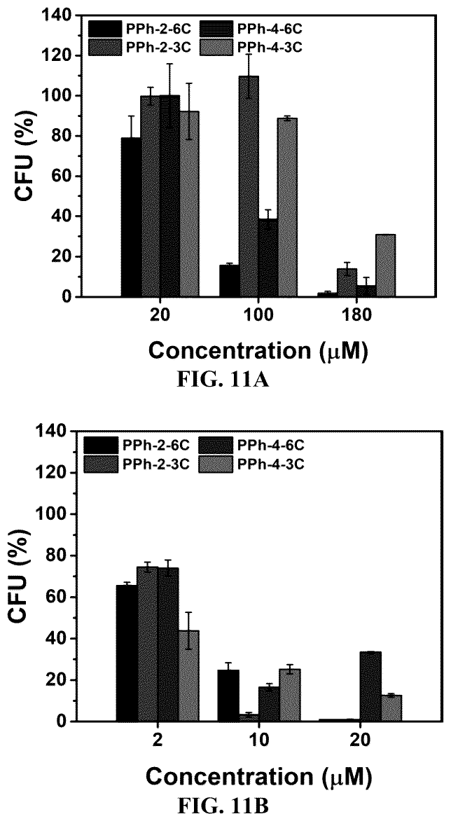

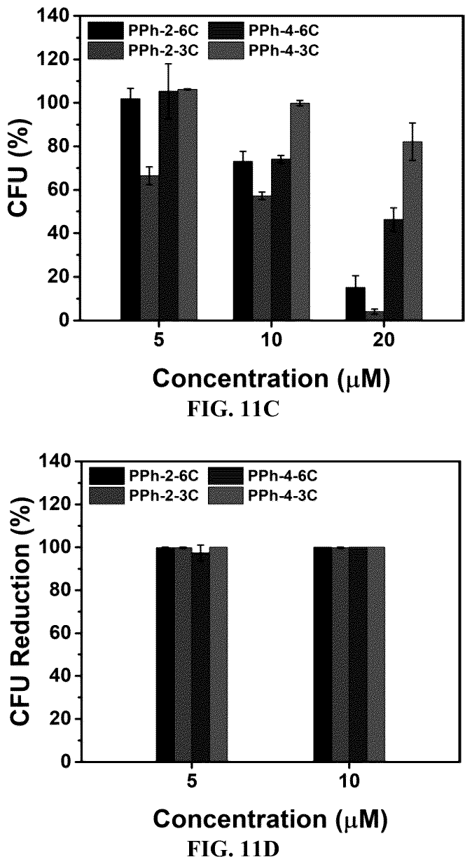

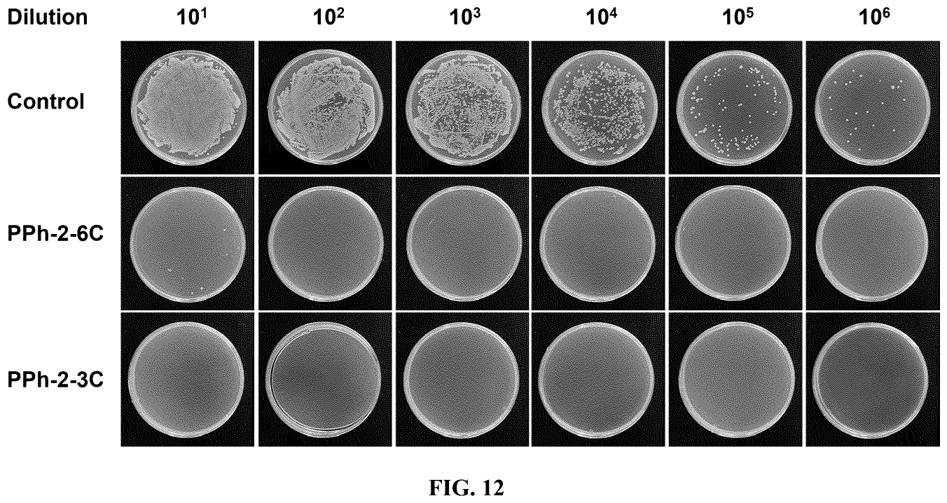

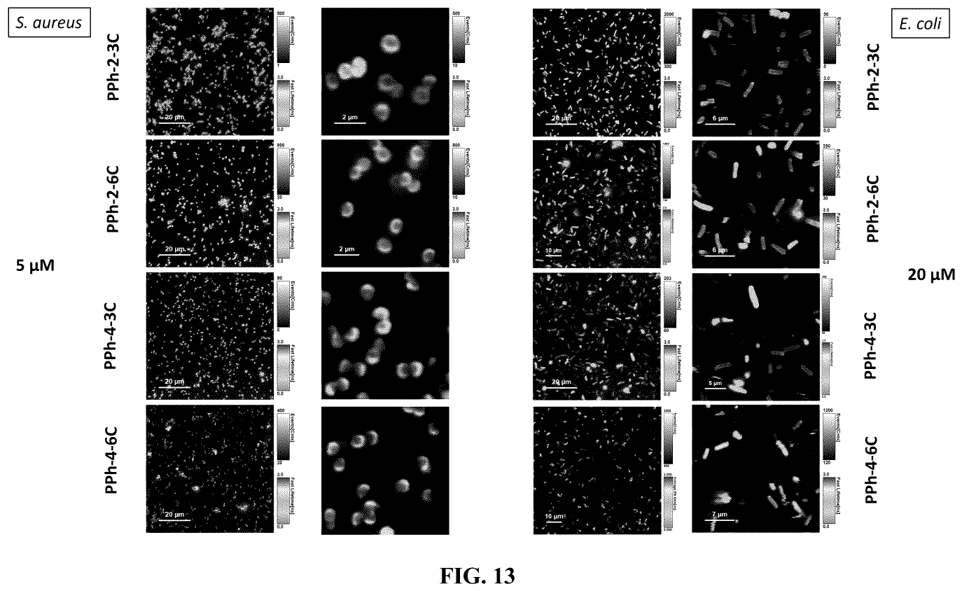

Water-soluble CPs are multifunctional materials that incorporate charged pendants groups to overcome limitations on solubility and processability. The tunability of the π-conjugated backbone and versatility of the charged side-chains allow water-soluble CPs to deliver enhanced light-harvesting features and greater compatibility with biological systems, especially, biocidal activity against Gram-positive and Gram-negative bacteria. The cationic pendant groups strongly interact with the bacterial cell membranes and generate ROS to photodynamically inactivate bacterial strains (FIG. 7). Latest reports include CPs with typical quaternary ammonium and imidazolium pendant groups. Hladysh et al. first synthesized phosphonium-bearing polythiophenes. More recently, Lichon et al. described the synthesis and usage of CPs with phosphonium groups for imaging and phototherapy. These initial efforts on designing novel phosphonium-containing CPs serve as launching platform for materials used for phototherapeutic applications.

As antibacterial treatments progress, conjugated polyelectrolytes (CPEs) have displayed intriguing therapeutic features to contribute to bridging the gap between drug-resistant pathogens and novel effective antibiotic materials. The current disclosure describes the light-activated antibacterial activity of a new class of phosphonium-containing conjugated polyelectrolytes. Previously, these polymers have been demonstrated to undergo the photosensitization of molecular oxygen into singlet oxygen and reactive oxygen species, a key factor for the photoinduced inactivation of bacteria. As a result, in vitro antibacterial assays against Gram-negative Escherichia coli and Gram-positive Staphylococcus aureus were performed employing the series of polyelectrolytes under both dark and illumination conditions. The main findings suggest that the structural features of the polymers, such as the length of the side chains, influence the antibacterial properties. The polyelectrolytes with longer side chains show greater inhibition on E. coli bacterial cells due to enhanced structural amphiphilic balance. For S. aureus, all polymers were highly active at low concentrations. Furthermore, cell culture with serial dilutions was completed to quantitatively assess the strength of the light-activated antibacterial activity, yielding significant log reduction values which reached to 6.11. To comprehend the polymer-bacteria interactions, confocal fluorescence microscopy imaging was employed. Remarkably, the polyelectrolytes with longer alkyl chains locate inside the E. coli bacterial cells. These polymers offer a new approach in the application of phosphonium-containing PPE-based conjugated polyelectrolytes to photodynamically inactivating pathogenic resistant bacterial strains of public health pertinence.

### I. PHOSPHONIUM-CONTAINING CONJUGATED POLYELECTROLYTES

Poly(phenylene ethynylenes) (PPEs) are polymers that have a wide range of applications in electrically conducting materials, bio-chemical sensors, and supramolecular assemblies. The present disclosure provides a plurality of compounds generally referred to herein as poly (phenylene ethynylene) conjugates (PPE-conjugates, i.e., conjugated polymers with an attached or conjugated ligand), methods of synthesizing PPE conjugates and various uses for the PPE conjugates. The term conjugated polymer refers to the base polymer while the term polymer conjugate refers to the base polymer with an attached ligand.

In certain aspects a poly(phenylene ethynylene) as described herein can have a general structure of Formula Ia with n being between 2 to 500. In certain aspects the phenyl group can be substituted phenyl groups.

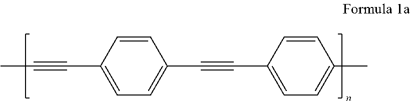

Certain embodiments are directed to phosphonium-functionalized conjugated polyelectrolytes, e.g., PPh-2-3C, PPh-2-6C, PPh-4-3C, PPh4-6C, see structures below where n can be any whole number between 2 and 500.

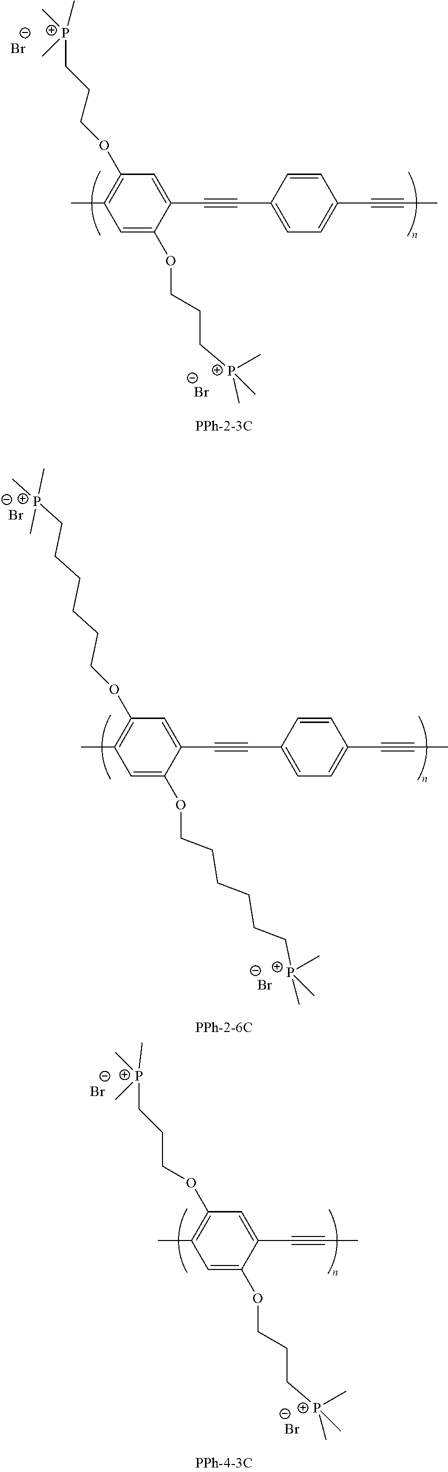

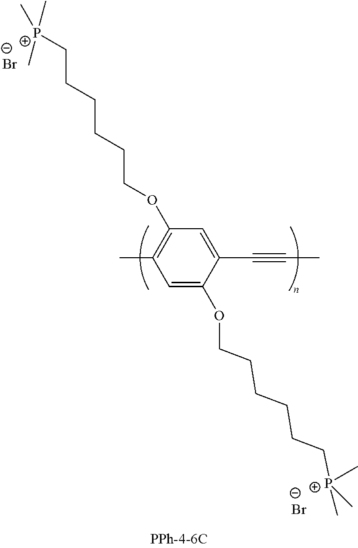

According to various embodiments the present disclosure provides phosphonium-functionalized conjugated polyelectrolytes having light activated biocidal activity, methods for making them, and methods of using them as biocidal materials. According to an embodiment, phosphonium-functionalized conjugated polyelectrolytes were synthesized and tested for biocidal activity, and were found to be effective in killing the bacteria. The above-mentioned compounds absorb light and can be effective biocidal agents for decontamination of media bearing populations of microorganisms, such as pathogenic bacteria.

The term “under illumination”, as used herein, refers to illumination of a biocidal oligomer of the invention, such as in contact with a microorganism, or a surface contaminated with microorganisms, such as bacteria, with actinic radiation, i.e., visible or ultraviolet (UV) light. It is understood that such illumination can be carried out in the presence of oxygen, such as in atmospheric air, to provide the oxygen which is electronically excited into its singlet state. “Singlet oxygen”, as is well known in the art, refers to the first electronic excited state of molecular oxygen O₂, which is a ground state triplet (i.e., two electrons with parallel spins), and can be excited into a singlet state (i.e., two electrons with antiparallel spins).

“Biocidal” activity, as the term is used herein, refers to action of the inventive compounds on living microorganisms whereby the compounds kill, block replication, control the population, or inhibit proliferation of the microorganisms, such as bacteria. It is believed that singlet oxygen can also be effective against other microorganisms, such as fungi, or against viral particles, or against bacterial or fungal spores. Biocidal activity can also occur versus eukaryotic organisms such as protozoans, and against multicellular organisms such as nematodes, insect larvae, the eggs of multicellular organisms, and the like. Due to the highly reactive nature of singlet oxygen, it is believed that a wide range of living or quasi-living (viral, spores, eggs, etc.) entities can be damaged or destroyed by use of compounds described herein in the presence of illumination.

“Decontamination” of a material refers to the effect of biocidal activity on a population of target microorganisms or other living or quasi-living entities disposed on or within the material.

There has been an increasing interest in the prevention of staphylococcal infections because of the increasing number of infections caused by methicillin-resistant S. aureus (MRSA), which is the most common cause of hospital-acquired infections.

### II. USES OF COMPOSITIONS OF THE INVENTION

In various embodiments, the invention provides a method of killing a microorganism or attenuating a population of the microorganism, comprising contacting the microorganism or the population thereof with an effective amount or concentration of a compound of the invention, optionally under illumination, such as with visible or UV light, such as in atmospheric air or other conditions where oxygen is present. In some embodiments, compounds of the invention can be photoactive, photosensitive, photodynamic, or photoresponsive. The compound can be used for photodynamic therapy, wherein the compound can be a photosensitizer and lead to the generation of, for example, singlet oxygen and reactive oxygen species (ROS). Wavelengths of light that can be used in a method of the invention include, for example, about 200 nm, about 210 nm, about 220 nm, about 230 nm, about 240 nm, about 250 nm, about 260 nm, about 270 nm, about 280 nm, about 290 nm, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 510 nm, about 520 nm, about 530 nm, about 540 nm, about 550 nm, about 560 nm, about 570 nm, about 580 nm, about 590 nm, about 600 nm, about 610 nm, about 620 nm, about 630 nm, about 640 nm, about 650 nm, about 660 nm, about 670 nm, about 680 nm, about 690 nm, about 700 nm, about 710 nm, about 720 nm, about 730 nm, about 740 nm, about 750 nm, about 760 nm, about 770 nm, about 780 nm, about 790 nm, and about 800 nm.

Activation of the compound can occur via exposure to light wherein the administration of the light is continuous or pulsed. Pulses of light can be separated by, for example, about 1 second, about 2 seconds, about 3 seconds, about 4 seconds, about 5 seconds, about 6 seconds, about 7 seconds, about 8 seconds, about 9 seconds, about 10 seconds, about 15 seconds, about 20 seconds, about 25 seconds, about 30 seconds, about 35 seconds, about 40 seconds, about 45 seconds, about 50 seconds, about 55 seconds, about 1 minute, about 1.5 minutes, about 2 minutes, about 2.5 minutes, about 3 minutes, about 3.5 minutes, about 4 minutes, about 4.5 minutes, about 5 minutes, about 6 minutes, about 7 minutes, about 8 minutes, about 9 minutes, about 10 minutes, about 15 minutes, or about 20 minutes.

In some embodiments, activation of the compound via light can occur concurrently with, or subsequent to, administration of the compound to a subject or location or surface. Light can then be administered to the subject or location or surface, for example, every 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 18 hours, 1 day, 2 days, 3 days, 4 days, 5 days, 6 days, or 1 week to improve efficacy of the compound.

The energy of light used to activate the compound can be, for example, about 10 J/cm², about 15 J/cm², about 20 J/cm², about 25 J/cm², about 30 J/cm², about 35 J/cm², about 40 J/cm², about 45 J/cm², about 50 J/cm², about 55 J/cm², about 60 J/cm², about 65 J/cm², about 66 J/cm², about 67 J/cm², about 68 J/cm², about 69 J/cm², about 70 J/cm², about 71 J/cm², about 72 J/cm², about 73 J/cm², about 74 J/cm², about 75 J/cm², about 76 J/cm², about 77 J/cm², about 78 J/cm², about 79 J/cm², about 80 J/cm², about 81 J/cm², about 82 J/cm², about 83 J/cm², about 84 J/cm², about 85 J/cm², about 86 J/cm², about 87 J/cm², about 88 J/cm², about 89 J/cm², about 90 J/cm², about 95 J/cm², or about 100 J/cm².

The brightness of light used to activate the compound can be, for example, about 100 lm, about 110 lm, about 120 lm, about 130 lm, about 140 lm, about 150 lm, about 160 lm, about 170 lm, about 180 lm, about 190 lm, about 200 lm, about 250 lm, about 300 lm, about 350 lm, about 450 lm, about 500 lm, about 550 lm, about 600 lm, about 650 lm, about 700 lm, about 750 lm, about 800 lm, about 850 lm, about 900 lm, about 950 lm, about 1000 lm, about 1100 lm, about 1200 lm, about 1300 lm, about 1400 lm, about 1500 lm, about 1600 lm, about 1700 lm, about 1800 lm, about 1900 lm, about 2000 lm, about 2500 lm, about 3000 lm, about 3500 lm, or about 4000 lm.

Bacterial strains that can be treated by a method described herein can be gram-negative or gram-positive. Non-limiting examples of microbes that can be treated by a method of the invention include Acinetobacter baumannii, carbapenem-resistant Enterobacteriaceae (CRE), clindamycin-resistant Group B Streptococcus, Clostridium difficile, drug-resistant Campylobacter, drug-resistant Neisseria gonorrhoeae, drug-resistant non-typhoidal Salmonella, drug-resistant Salmonella typhi, drug-resistant Shigella, drug-resistant Streptococcus pneumoniae, drug-resistant tuberculosis, erythromycin-resistant Group A Streptococcus, Escherichia coli, extended spectrum β-lactamase producing Enterobacteriaceae (ESBLs), fluconazole-resistant Candida, methicillin-resistant S. aureus (MRSA), multidrug-resistant Acinetobacter, multidrug-resistant Pseudomonas aeruginosa, S. aureus, VRE, and vancomycin-resistant S. aureus (VRSA). In some embodiments, the methods of the invention can be applied to agricultural pathogens.

In some embodiments, a therapy of the disclosure has synergistic activity in combination with an antibiotic. Synergy can refer to the observation that the combination of two therapeutic agents can have an overall effect that is greater than the sum of the two individual effects. Synergy can also refer to the observation that a single drug produces no effect but, when administered with a second drug produces an effect that is greater than the effect produced by the second therapeutic agent alone.

Classes of antibiotics that can be used in a method of invention include, for example, aminoglycosides, ansamycins, β-lactams, carbapenems, cephalosporins, glycopeptides, lincosamides, lipopeptides, macrolides, monobactams, nitrofurans, oxazolidinones, penicillins, polypeptides, quinolones, fluroquinolones, sulfonamides, and/or tetracyclines.

Non-limiting examples of antibiotics that can be used in a method of the invention include ampicillin, amoxicillin, azithromycin, carbenicillin, clarithromycin, dicloxicillin, doxycycline, erythromycin, gentamicin, kanamycin, methicillin, neomycin, norfloxacin, oxacillin, PMB, colisitin, penicillin, penicillin G, penicillin V, streptomycin, tetracycline, tobramycin, polyethyleneimine, lactic acid, benzoic acid bacitracin, imipenem, and vancomycin.

In some embodiments, compounds of the invention can be used to treat a condition caused by a microbe in a subject. In some embodiments, the microbe can be a bacterium, fungus, or protozoa.

For example, the invention can provide a method of decontaminating a surface contaminated with or at risk of contamination with a microorganism or a population thereof, comprising contacting the microorganism or the population thereof disposed on the contaminated surface with an effective amount or concentration of a compound of the invention, optionally under illumination. As described above, a population of microorganisms, such as pathogenic bacteria, disposed on a surface, such as the surface of a surgical instrument or facility, or a sterile surface in a processing facility, can be decontaminated by exposing the population to light in the presence of an effective level of a biocidal oligomer of the invention. The surface can be virtually any material, such as metal, glass, ceramic, plastic, or the like, provided that the surface can be illuminated.

For example, the invention can provide a method of decontaminating a fabric contaminated with a microorganism or a population thereof, comprising contacting the microorganism or the population thereof disposed on or within fibers of the contaminated fabric with an effective amount or concentration of a compound of the invention, optionally under illumination. A fabric, consisting of fibers, can harbor populations of pathogenic bacteria disposed on and within the fibers. Exposure of the bacteria, such as by soaking the fabric in a water or alcohol solution of a biocidal oligomer of the invention, concurrently with or prior to exposure to visible or UV light, can be used to effectively decontaminate the fabric. Fabrics such as bed sheets, bandages, clothing, and the like can be decontaminated using the biocidal oligomers of the invention in conjunction with illumination.

For example, the invention can provide a method of decontaminating a transparent liquid contaminated with a microorganism or a population thereof, comprising contacting the microorganism or the population thereof disposed within the transparent liquid with an effective amount or concentration of a compound of the invention, optionally under illumination. For expression of the illumination-enhanced biocidal activity of the compound, the microorganism in contact with the biocidal compound can be exposed to levels of illumination sufficient to induce formation of toxic levels of singlet oxygen in the vicinity of the microorganism. Compositions of the invention can thus be used to kill bacterial populations in, for example, water that is to be used for washing purposes. If it is desired to subsequently remove the biocidal compounds from the water, the ionic nature of the compounds can be used to remove them from the water using an ion-exchange resin or the like.

For example, the invention can provide a kit for decontamination of a surface, a fabric, or a liquid or transparent liquid, comprising a composition including the compound of the invention, optionally in a suitable solvent or medium; and, optionally, a source of illumination; optionally comprising additional biocidal materials; and optionally including instructions for use. For decontamination of objects or materials, capable of being exposed to illumination, a kit can be provided including a biocidal oligomer of the invention, such as in water or alcohol solution, in conjunction with instructions for use. A light source (visible or UV) can also be included in the kit; alternatively, sunlight or artificial illumination can be used as a light source. The solution containing the biocidal oligomer can further contain other sterilizing ingredients, such as surfactants, provided they do not react with the inventive oligomer. The chemical stability of the inventive oligomers, which is believed to be of a high degree due to the robust chemical structures, can make them compatible with a wide range of other sterilant materials. Or, the kit can include a second container with a sterilant, such as an oxidizing agent, a halogen, or the like, for separate application to the object or material to be sterilized.

### III. PHARMACEUTICAL COMPOSITIONS OF THE INVENTION

A pharmaceutical composition of the invention can be used, for example, before, during, or after treatment of a subject with light, antibiotics, or another pharmaceutical agent. A pharmaceutical composition of the invention can be a combination of a phosphonium-containing conjugated polyelectrolyte compounds described herein with other chemical components, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration.

A pharmaceutical composition can be administered in a local manner, for example, via injection of the compound directly into an organ, optionally in a depot or sustained release formulation or implant. Pharmaceutical compositions can be provided in the form of a rapid release formulation, in the form of an extended release formulation, or in the form of an intermediate release formulation. A rapid release form can provide an immediate release. An extended release formulation can provide a controlled release or a sustained delayed release.

For oral administration, pharmaceutical compositions can be formulated by combining the active compounds with pharmaceutically-acceptable carriers or excipients. Such carriers can be used to formulate liquids, gels, syrups, elixirs, slurries, or suspensions, for oral ingestion by a subject. Non-limiting examples of solvents used in an oral dissolvable formulation can include water, ethanol, isopropanol, saline, physiological saline, DMSO, dimethylformamide, potassium phosphate buffer, phosphate buffer saline (PBS), sodium phosphate buffer, 4-2-hydroxyethyl-1-piperazineethanesulfonic acid buffer (HEPES), 3-(N-morpholino) propanesulfonic acid buffer (MOPS), piperazine-N,N′-bis(2-ethanesulfonic acid) buffer (PIPES), and saline sodium citrate buffer (SSC). Non-limiting examples of co-solvents used in an oral dissolvable formulation can include sucrose, urea, cremaphor, DMSO, and potassium phosphate buffer.

Pharmaceutical preparations can be formulated for intravenous administration. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of the active compounds in water-soluble form. Suspensions of the active compounds can be prepared as oily injection suspensions. Suitable lipophilic solvents or vehicles include fatty oils such as sesame oil, or synthetic fatty acid esters, such as ethyl oleate or triglycerides, or liposomes. The suspension can also contain suitable stabilizers or agents which increase the solubility of the compounds to allow for the preparation of highly concentrated solutions. Alternatively, the active ingredient can be in powder form for constitution with a suitable vehicle, e.g., sterile pyrogen-free water, before use.

The active compounds can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers and preservatives.

The compounds can also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, and PEG. In suppository forms of the compositions, a low-melting wax such as a mixture of fatty acid glycerides, optionally in combination with cocoa butter, can be melted.

In practicing the methods of treatment or use provided herein, therapeutically-effective amounts of the compounds described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compounds used, and other factors. The compounds can be used singly or in combination with one or more therapeutic agents as components of mixtures.

Pharmaceutical compositions can be formulated using one or more physiologically-acceptable carriers comprising excipients and auxiliaries, which facilitate processing of the active compounds into preparations that can be used pharmaceutically. Formulation can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising the compounds described herein include formulating the compounds with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the invention include liquid, powder, gel, nanosuspension, nanoparticle, microgel, aqueous or oily suspensions, emulsion, and any combination thereof.

Non-limiting examples of pharmaceutically-acceptable excipients suitable for use in the invention include binding agents, disintegrating agents, anti-adherents, anti-static agents, surfactants, anti-oxidants, coating agents, coloring agents, plasticizers, preservatives, suspending agents, emulsifying agents, anti-microbial agents, spheronization agents, and any combination thereof.

A composition of the invention can be, for example, an immediate release form or a controlled release formulation. An immediate release formulation can be formulated to allow the compounds to act rapidly. Non-limiting examples of immediate release formulations include readily dissolvable formulations. A controlled release formulation can be a pharmaceutical formulation that has been adapted such that release rates and release profiles of the active agent can be matched to physiological and chronotherapeutic requirements or, alternatively, has been formulated to effect release of an active agent at a programmed rate. Non-limiting examples of controlled release formulations include granules, delayed release granules, hydrogels (e.g., of synthetic or natural origin), other gelling agents (e.g., gel-forming dietary fibers), matrix-based formulations (e.g., formulations comprising a polymeric material having at least one active ingredient dispersed through), granules within a matrix, polymeric mixtures, and granular masses.

In some, a controlled release formulation is a delayed release form. A delayed release form can be formulated to delay a compound's action for an extended period of time. A delayed release form can be formulated to delay the release of an effective dose of one or more compounds, for example, for about 4, about 8, about 12, about 16, or about 24 hours.

A controlled release formulation can be a sustained release form. A sustained release form can be formulated to sustain, for example, the compound's action over an extended period of time. A sustained release form can be formulated to provide an effective dose of any compound described herein (e.g., provide a physiologically-effective blood profile) over about 4, about 8, about 12, about 16 or about 24 hours.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Multiple therapeutic agents can be administered in any order or simultaneously. In some embodiments, a compound of the invention is administered in combination with, before, or after an antibiotic. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

Therapeutic agents described herein can be administered before, during, or after the occurrence of a disease or condition, and the timing of administering the composition containing a therapeutic agent can vary. For example, the compositions can be used as a prophylactic and can be administered continuously to subjects with a propensity to conditions or diseases in order to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A therapeutic agent can be administered as soon as is practicable after the onset of a disease or condition is detected or suspected, and for a length of time necessary for the treatment of the disease, such as, for example, from about 1 month to about 3 months. The length of treatment can vary for each subject.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Non-limiting examples of pharmaceutically active agents suitable for combination with compositions of the disclosure include anti-infectives, i.e., aminoglycosides, antiviral agents, antimicrobials, anticholinergics/antispasmotics, antidiabetic agents, antihypertensive agents, antineoplastics, cardiovascular agents, central nervous system agents, coagulation modifiers, hormones, immunologic agents, immunosuppressive agents, and ophthalmic preparations.

Compounds can be delivered via liposomal technology. The use of liposomes as drug carriers can increase the therapeutic index of the compounds. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues. Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

Compositions of the invention can be packaged as a kit. In some embodiments, a kit includes written instructions on the administration/use of the composition. The written material can be, for example, a label. The written material can suggest conditions methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### IV. DOSING

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein can be present in a composition in a range of from about 1 mg to about 2000 mg; from about 100 mg to about 2000 mg; from about 10 mg to about 2000 mg; from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a dose can be expressed in terms of an amount of the drug divided by the mass of the subject, for example, milligrams of drug per kilograms of subject body mass. In some embodiments, a compound is administered in an amount ranging from about 5 mg/kg to about 50 mg/kg, 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg.

### V. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Cationic Water-Soluble Conjugated Polymers: Photophysical and Antibacterial Properties

### A. Results

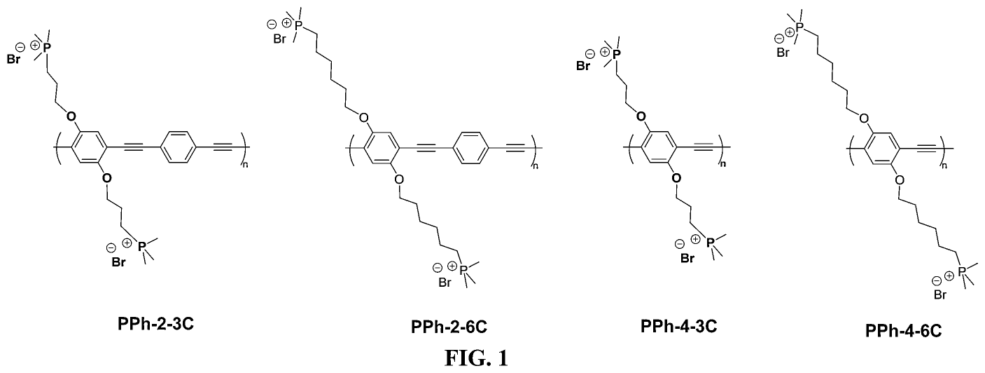

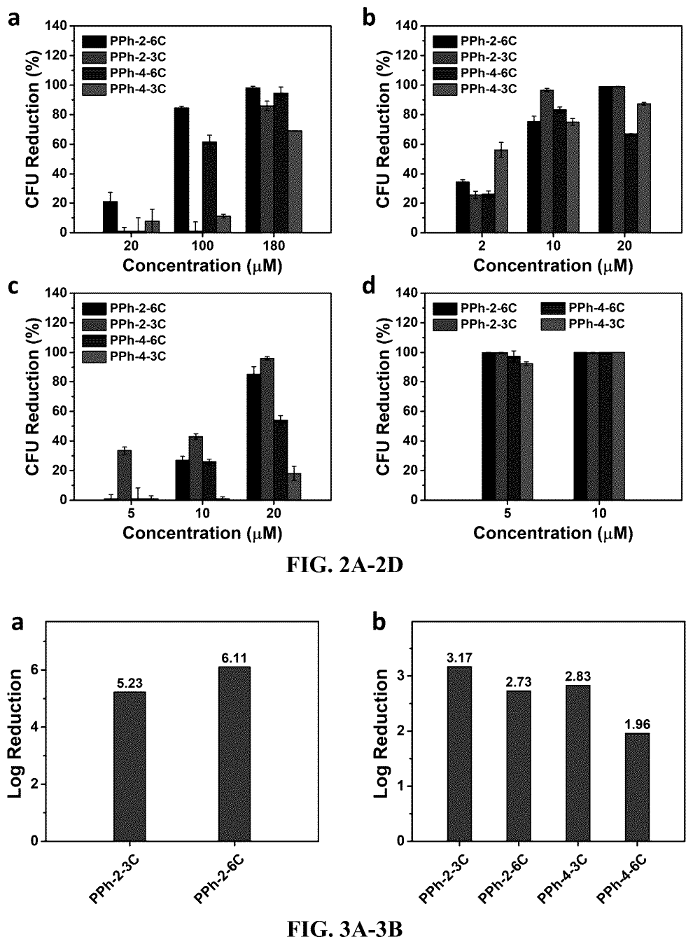

Antibacterial Activity of Conjugated Polyelectrolytes. The antibacterial activity of the phosphonium-functionalized conjugated polyelectrolytes was evaluated against the E. coli (ATCC 25922) strain and S. aureus (ATCC 29213) strain as two model strains of Gram-positive and Gram-negative bacteria, respectively. Bacterial survival assays were performed in the absence and presence of light irradiation conditions using a traditional plate count method. First, to assess the dark toxicity of the phosphonium-functionalized conjugated polyelectrolytes shown in FIG. 1, bacteria solutions were treated with varying material concentrations (5-180 μM). The concentration of the polymers was calculated based on the molar mass of the polymer repeat units (PRUs). As shown in FIG. 2a and FIG. 2c, increasing the concentration of polymers improved the antibacterial activity against E. coli. Furthermore, PPh-2-6C and PPh-4-6C showed enhanced antibacterial activity compared to PPh-2-3C and PPh-4-3C at concentrations of 100 μM and 180 μM (FIG. 2a), indicating the hydrophobic length segment affects the inhibition effect against E. coli and the polymers with hexyl groups were more effective than those with propyl groups. This might be because the increase in the hydrophobicity of the polymer structure enhances their hydrophobic interactions with the phospholipid bilayer of E. coli. Thus, resulting in improved antibacterial activity. Note that the structure of the cellular envelope is different between Gram-positive and Gram-negative bacteria. Gram-negative bacteria have an outer membrane containing phospholipid bilayer and lipopolysaccharide, which acts as a barrier against the polymeric biocides, while Gram-positive bacteria lack an outer membrane but are surrounded by layers of a peptidoglycan cell wall, facilitating the penetration of antimicrobial polymers through it and interaction with the membrane. Therefore, Gram-positive bacteria typically displayed lower resistance to antimicrobial polymers than Gram-negative ones. As expected, all four polymers exhibited antibacterial effects at a relatively low concentration of 20 μM against S. aureus (killing efficiency >66%). In contrast, these polymers barely showed antibacterial activity against E. coli at the same concentration (killing efficiency <23%).

Previous work has found that the phosphonium-functionalized poly(phenylene ethynylene)-type conjugated polyelectrolytes exhibited excellent performance as singlet oxygen photosensitizers, and the efficient production of ROS was detected under visible-light irradiation. The light-activated antibacterial properties of these four polymers were investigated. The polymer-treated bacteria were irradiated with a 400 nm LED lamp for 15 min (the light irradiance was 16 mW/cm²). As shown in FIG. 2B, PPh-2-6C and PPh-2-3C exhibited killing activity against E. coli above 99% at the concentration of 20 μM. In FIG. 2D, all four polymers displayed killing activity against S. aureus above 99% at the concentration of 10 μM. These data demonstrate significant bacterial-killing efficacy of the conjugated polyelectrolytes under exposure to light. Moreover, the light dose (14.4 J cm-2) employed in the experiment was much less than those of reported analogs photodynamic antimicrobial systems.³²⁻³⁴

Quantitative Evaluation of Light-Induced Antibacterial Activity. To obtain a better and more reliable quantitative light-activated killing in the 99% and higher ranges, cell plating techniques were used to measure cell colony forming units (CFU) by incubation of aliquots of antimicrobial-treated planktonic bacteria on agar plates and with serial dilution from 1×10⁻¹ to 1×10⁻⁶ based on the previously reported method.³⁵ The colonies observed in the controls and the samples treated with polymers under low dilution fold are too numerous to be accurately counted. As successive dilution reduces the number of CFUs, a range of countable colonies becomes discernible. The log reduction of CFU is defined as Log N_{control}−Log Nₛₐₘₚₗₑ=Log(N_{control}/Nₛₐₘₚₗₑ), and the calculated results are displayed in FIG. 3. We observed >5 log kills against E. coli for PPh-2-6C and PPh-2-3C under irradiation at the concentration of 20 μM (FIG. 3A). For S. aureus, the log kills of PPh-2-3C, PPh-2-6C, PPh-4-3C, and PPh-4-6C at the concentration of 10 μM are 3.17, 2.73, 2.83, and 1.96 respectively.

Antibacterial Mechanisms. To investigate the interaction mechanism between polymers and bacteria, confocal microscopy was utilized to directly visualize the binding of polymers to E. coli and S. aureus. FIG. 4 shows that with an increase in treatment time and polymer concentration, more E. coli bacteria were stained by the polymers. After 30 minutes of incubation at a concentration of 20 μM, all four polymers attached to E. coli. To get more information, the bacteria were zoomed out and most of the bacteria in the image were focused on the middle layer for a clearer observation of the location of polymers. As shown in FIG. 4, E. coli treated with PPh-2-3C and PPh-4-3C displayed high fluorescence intensity on the membrane and low intensity inside of bacteria, indicating that PPh-2-3C and PPh-4-3C mainly attached on the surface of E. coli rather than entering the cells. In contrast, E. coli treated with PPh-2-6C and PPh-4-6C exhibited higher fluorescence signals inside of cells, referring that PPh-2-6C and PPh-4-6C are located inside the cells. In addition, E. coli treated with PPh-4-6C exhibited different fluorescence lifetimes from the inside of the cell and cell surface according to the fluorescence lifetime maps. The polymers inside of the bacteria showed a longer fluorescence lifetime, while the polymers attached to the membrane showed a relatively lower fluorescence lifetime. This might be because the high viscosity inside of bacteria reduced the vibration of polymers, resulting in enhanced fluorescence intensity. The Z-scan analysis of E. coli in different depths also demonstrated that PPh-2-6C and PPh-4-6C were mainly located inside of cells and PPh-2-3C and PPh-4-3C just bind on the surface of cells. Comparing to the propyl groups, the hexyl groups in the sidechains of PPh-2-6C and PPh-4-6C are more prone to disrupt the membrane integrity of E. coli, thus, causing improved dark-toxicity than that of PPh-2-3C and PPh-4-3C.

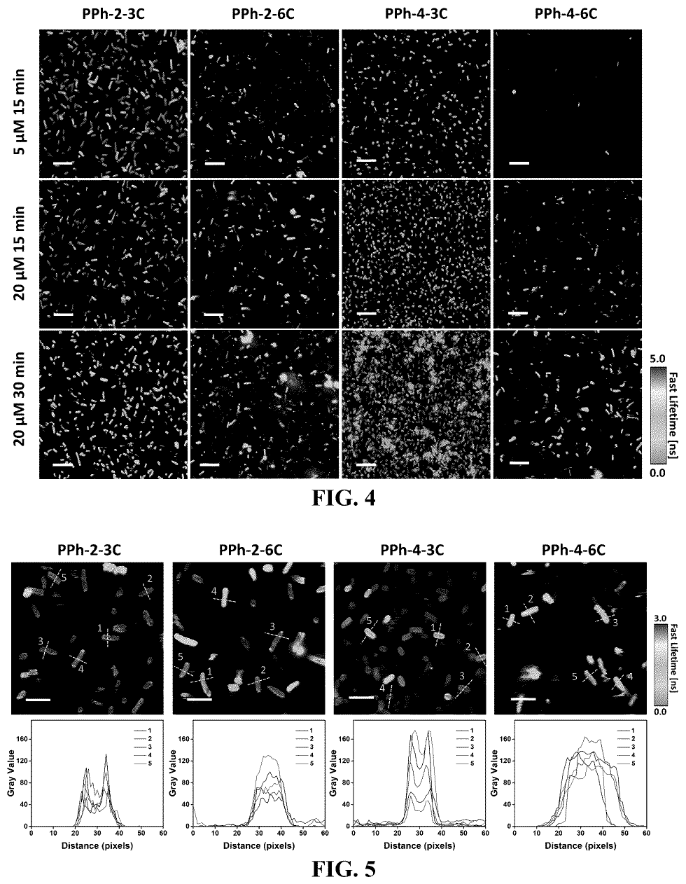

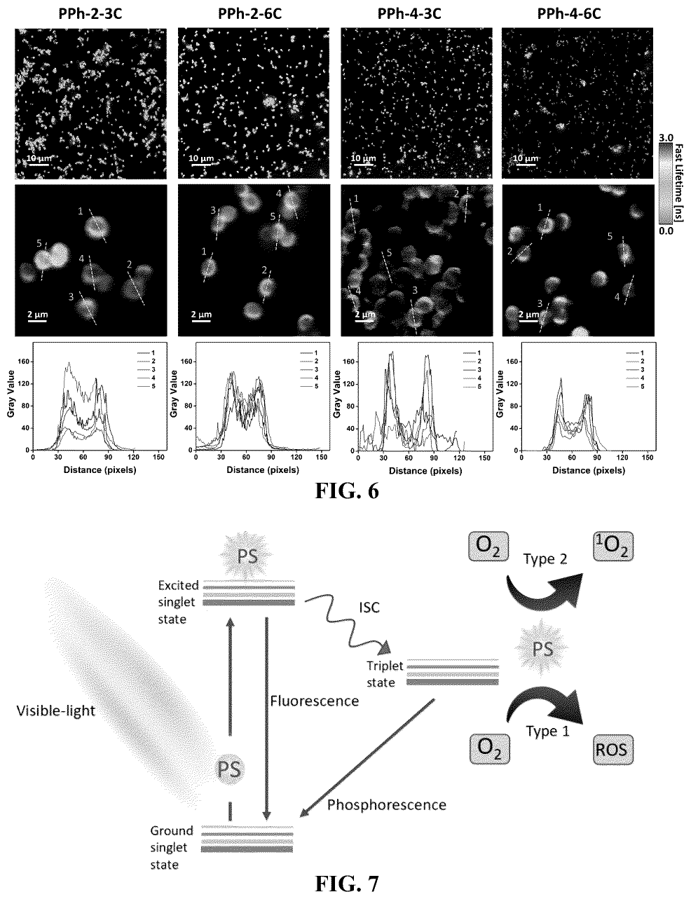

Due to the high sensitivity of S. aureus toward polymers, the microscope images for polymer treated S. aureus were taken at a relatively low polymer concentration (5 μM) and a short incubation time (15 min). As shown in FIG. 5, all four polymers bind to S. aureus, and the zoomed images exhibited that the polymers mainly attached to the surface of S. aureus. This might occur as a result of electrostatic interactions between the polymers and S. aureus, which allowed the polymeric chains to intercalate into the thick, heavily crosslinked, porous peptidoglycan cell wall.

The light-activated antibacterial properties of a series of four newly reported phosphonium-containing conjugated polyelectrolytes against E. coli and S. aureus bacterial strains were studied. Earlier investigations proved the efficient formation of the triplet excited state of the polymers under visible-light irradiation to yield highly reactive oxygen species. The series of polyelectrolytes were therefore considered potential light-induced bactericidal agents. The usage of these polymers as photosensitizing materials was found to lead to a significant reduction of bacterial colonies, existing dependence of the efficacy on the polymeric structural characteristics. For instance, PPh-2-6C and PPh-4-6C in the absence of light displayed an enhanced antibacterial activity against E. coli due to the increase of interactions between the longer hydrophobic segments of the pendant groups and the bacterial cells. Under light exposure, PPh-2-3C and PPh-2-6C at 20 μM revealed improved antibacterial activity above 99% of inhibition rate and >5 log kills against E. coli. In addition, confocal microscopy imaging served as a tool to identify the mechanistic uptake of polymers into the bacterial cells and to clarify the binding affinity to the cell membrane based on the structural differences of the polymers. The main findings confirmed that the polymers can act as efficient light-harvesting and photosensitizing agents to generate oxygen-derived free radicals and to cause bacterial oxidative stress and cell damage when exposed to light. In general, the results suggest that the set of polymers effectively renders E. coli and S. aureus bacterial strains inactive at low light doses. Although it is true the antibacterial activity rises from the cationic stress and oxidative damage of ROS, secondary mechanisms are likely to occur to contribute to the synergistic biocidal activity. As part of innovative applications to minimize the detrimental effects of pathogenic diseases on public health, we envision that the set of polymers presented herein open a new venue to conduct further research on the development of conjugated polymeric materials with phosphonium functionality.

Molecular Weight determination. PPh-2-6C and PPh-4-6C display a red shift in the absorption spectra. The extended alkyl side chains enable greater intramolecular separation of the cationic groups, therefore, the planarity and molecular orbital overlapping of the conjugated backbone is affected to a lesser extent compared to PPh-2-3C and PPh-4-3C. The emission band broadening, low quantum yields, and short lifetimes evidence the interchain aggregation of the CPs in water.

Transient absorption spectroscopy. Pump-probe transient absorption experiments proved the presence of a long-lived component attributed to the triplet excited state formed by intersystem crossing.

¹O₂ emission and reactive oxygen species. The steady-state near-infrared luminescent at 1270 nm confirmed the photosensitized production of singlet oxygen. PPh-2-3C and PPh-4-3C with shorter side-chains showed greater ROS generation due to the efficient 102 diffusion to react with the ADMA probe before decaying.

Antibacterial activity. Results suggest the biocidal activity is higher after light irradiation (λ=400 nm, 15 min, 16 mW/cm²). The greater antibacterial efficiency of PPh-2-3C and PPh-2-6C could be attributed to the enhanced amphiphilic balance. Oxidative stress induces the degradation of the bacterial membrane and intracellular components.

### B. Methods

Methods used in the studies include Diffusion-ordered NMR Spectroscopy for molecular weight characterization using PEG standards for external calibration; Steady and time-resolved fluorescence spectroscopy analysis; Femtosecond and nanosecond transient absorption spectroscopy; Singlet oxygen and reactive oxygen species determination; Antibacterial activity measured by count plate method; Confocal Laser Scanning Microscopy (CLSM).

### REFERENCES

(1) Chinemerem Nwobodo, D.; Ugwu, M. C.; Oliseloke Anie, C.; Al-Ouqaili, M. T. S.; Chinedu Ikem, J.; Victor Chigozie, U.; Saki, M. Antibiotic Resistance: The Challenges and Some Emerging Strategies for Tackling a Global Menace. Journal of Clinical Laboratory Analysis. John Wiley and Sons Inc Sep. 1, 2022.
(2) Antibiotic Resistance Threats in the United States, 2019; Atlanta, Georgia, 2019.
(3) Global Antimicrobial Resistance and Use Surveillance System (GLASS) Report 2022; 2022.
(4) Kourtis, A. P.; Sheriff, E. A.; Weiner-Lastinger, L. M.; Elmore, K.; Preston, L. E.; Dudeck, M.; McDonald, L. C. Antibiotic Multidrug Resistance of Escherichia coli Causing Device- and Procedure-Related Infections in the United States Reported to the National Healthcare Safety Network, 2013-2017. Clinical Infectious Diseases 2021, 73 (11), E4552-E4559.
(5) Kourtis, A. P.; Hatfield; Kelly; Baggs, J.; Mu; Yi; See, I.; Epson, E.; Nadle; Joelle; Kainer, M. A.; Dumyati, G.; Petit, S.; Ray, S. M.; Ham, D.; Capers, C.; Ewing; Heather; Coffin, N.; Mcdonald; L Clifford; Jernigan, J.; Cardo, D. Morbidity and Mortality Weekly Report Vital Signs: Epidemiology and Recent Trends in Methicillin-Resistant and in Methicillin-Susceptible Staphylococcus Aureus Bloodstream Infections-United States; 2017.
(6) Li, Z.; Lu, W.; Jia, S.; Yuan, H. Backbone-Regulated Cationic Conjugated Polymers for Combating and Monitoring Pathogenic Bacteria. ACS Appl Polym Mater 2022, 4 (1), 29-35.
(7) Cui, Q.; Yuan, H.; Bao, X.; Ma, G.; Wu, M.; Xing, C. Synergistic Photodynamic and Photothermal Antibacterial Therapy Based on a Conjugated Polymer Nanoparticle-Doped Hydrogel. ACS Appl Bio Mater 2020, 3 (7), 4436-4443.
(8) Monge, F. A.; Jagadesan, P.; Bondu, V.; Donabedian, P. L.; Ista, L.; Chi, E. Y.; Schanze, K. S.; Whitten, D. G.; Kell, A. M. Highly Effective Inactivation of SARS-COV-2 by Conjugated Polymers and Oligomers. ACS Appl Mater Interfaces 2020, 12 (50), 55688-55695.
(9) Sun, H.; Martinez, D.; Li, Z.; Schanze, K. S. Biofunctionalization of Water-Soluble Poly(PhenyleneEthynylene)s. ACS Appl Mater Interfaces 2020, 12 (47), 53310-53317.
(10) Sun, H.; Schanze, K. S. Functionalization of Water-Soluble Conjugated Polymers for Bioapplications. ACS Appl Mater Interfaces 2022.
(11) Li, Z.; Lu, W.; Jia, S.; Yuan, H.; Gao, L. H. Design and Application of Conjugated Polymer Nanomaterials for Detection and Inactivation of Pathogenic Microbes. ACS Applied Bio Materials. American Chemical Society Jan. 18, 2021, pp 370-386.
(12) Liang, J.; Wu, P.; Tan, C.; Jiang, Y. White Light-Induced Cell Apoptosis by a Conjugated Polyelectrolyte through Singlet Oxygen Generation. RSC Adv 2018, 8 (17), 9218-9222.
(13) Chemburu, S.; Corbitt, T. S.; Ista, L. K.; Ji, E.; Fulghum, J.; Lopez, G. P.; Ogawa, K.; Schanze, K. S.; Whitten, D. G. Light-Induced Biocidal Action of Conjugated Polyelectrolytes Supported on Colloids. Langmuir 2008, 24 (19), 11053-11062.
(14) Ji, E.; Corbitt, T. S.; Parthasarathy, A.; Schanze, K. S.; Whitten, D. G. Light and Dark-Activated Biocidal Activity of Conjugated Polyelectrolytes. ACS Appl Mater Interfaces 2011, 3 (8), 2820-2829.
(15) Wang, Y.; Jett, S. D.; Crum, J.; Schanze, K. S.; Chi, E. Y.; Whitten, D. G. Understanding the Dark and Light-Enhanced Bactericidal Action of Cationic Conjugated Polyelectrolytes and Oligomers. Langmuir 2013, 29 (2), 781-792.
(16) Wang, Y.; Corbitt, T. S.; Jett, S. D.; Tang, Y.; Schanze, K. S.; Chi, E. Y.; Whitten, D. G. Direct Visualization of Bactericidal Action of Cationic Conjugated Polyelectrolytes and Oligomers. Langmuir 2012, 28 (1), 65-70.
(17) Wang, Y.; Zhou, Z.; Zhu, J.; Tang, Y.; Canady, T. D.; Chi, E. Y.; Schanze, K. S.; Whitten, D. G. Dark Antimicrobial Mechanisms of Cationic Phenylene Ethynylene Polymers and Oligomers against Escherichia coli. Polymers (Basel) 2011, 3 (3), 1199-1214.
(18) Zhai, L.; Zhang, Z.; Zhao, Y.; Tang, Y. Efficient Antibacterial Performance and Effect of Structure on Property Based on Cationic Conjugated Polymers. Macromolecules 2018, 51 (18), 7239-7247.
(19) Xu, Q.; He, P.; Wang, J.; Chen, H.; Lv, F.; Liu, L.; Wang, S.; Yoon, J. Antimicrobial Activity of a Conjugated Polymer with Cationic Backbone. Dyes and Pigments 2019, 160, 519-523.
(20) Huang, Y.; Pappas, H. C.; Zhang, L.; Wang, S.; Cai, R.; Tan, W.; Wang, S.; Whitten, D. G.; Schanze, K. S. Selective Imaging and Inactivation of Bacteria over Mammalian Cells by Imidazolium-Substituted Polythiophene. Chemistry of Materials 2017, 29 (15), 6389-6395.
(21) Parthasarathy, A.; Pappas, H. C.; Hill, E. H.; Huang, Y.; Whitten, D. G.; Schanze, K. S. Conjugated Polyelectrolytes with Imidazolium Solubilizing Groups. Properties and Application to Photodynamic Inactivation of Bacteria. ACS Appl Mater Interfaces 2015, 7 (51), 28027-28034.
(22) Ji, E.; Parthasarathy, A.; Corbitt, T. S.; Schanze, K. S.; Whitten, D. G. Antibacterial Activity of Conjugated Polyelectrolytes with Variable Chain Lengths. Langmuir 2011, 27 (17), 10763-10769.
(23) Kanazawa, Ak.; Ikeda, Tom.; N D O, T. E. Polymeric Phosphonium Salts as a Novel Class of Cationic Biocides. IX. Effect of Side-Chain Length between Main Chain and Active Group on Antibacterial Activity.
(24) Hladysh, S.; Bondarev, D.; Svoboda, J.; Vohlídal, J.; Vrbata, D.; Zedník, J. Novel Conjugated Polyelectrolytes Based on Polythiophene Bearing Phosphonium Side Groups. Eur Polym J 2016, 83, 367-376.
(25) Hladysh, S.; Murmiliuk, A.; Vohlídal, J.; Havlíček, D.; Sedlařík, V.; Štěpánek, M.; Zedník, J. Combination of Phosphonium and Ammonium Pendant Groups in Cationic Conjugated Polyelectrolytes Based on Regioregular Poly(3-Hexylthiophene) Polymer Chains. Eur Polym J 2018, 100, 200-208.
(26) Hu, Z.; Zheng, N.; Dong, S.; Liu, X.; Chen, Z.; Ying, L.; Duan, C.; Huang, F.; Cao, Y. Phosphonium Conjugated Polyelectrolytes as Interface Materials for Efficient Polymer Solar Cells. Org Electron 2018, 57, 151-157.
(27) Zhang, C.; Du, K.; Zhang, X.; Zheng, X.; Cao, G.; Zhang, F.; Feng, F. Optical Properties of Phosphonium-, Quaternary Ammonium- and Imidazolium-Substituted Regioregular Polythiophenes and Application for Imaging Live Cells. Dyes and Pigments 2019, 170.
(28) Zhou, H.; Tang, D.; Kang, X.; Yuan, H.; Yu, Y.; Xiong, X.; Wu, N.; Chen, F.; Wang, X.; Xiao, H.; Zhou, D. Degradable Pseudo Conjugated Polymer Nanoparticles with NIR-II Photothermal Effect and Cationic Quaternary Phosphonium Structural Bacteriostasis for Anti-Infection Therapy. Advanced Science 2022, 9 (16).
(29) Cuthbert, T. J.; Hisey, B.; Harrison, T. D.; Trant, J. F.; Gillies, E. R.; Ragogna, P. J. Surprising Antibacterial Activity and Selectivity of Hydrophilic Polyphosphoniums Featuring Sugar and Hydroxy Substituents. Angewandte Chemie 2018, 130 (39), 12889-12892.
(30) Guo, A.; Wang, F.; Lin, W.; Xu, X.; Tang, T.; Shen, Y.; Guo, S. Evaluation of Antibacterial Activity of N-Phosphonium Chitosan as a Novel Polymeric Antibacterial Agent. Int J Biol Macromol 2014, 67, 163-171.
(31) Hisey, B.; Ragogna, P. J.; Gillies, E. R. Phosphonium-Functionalized Polymer Micelles with Intrinsic Antibacterial Activity. Biomacromolecules 2017, 18 (3), 914-923.
(32) Xiu, W.; Wan, L.; Yang, K.; Li, X.; Yuwen, L.; Dong, H.; Mou, Y.; Yang, D.; Wang, L. Potentiating Hypoxic Microenvironment for Antibiotic Activation by Photodynamic Therapy to Combat Bacterial Biofilm Infections. Nat Commun 2022, 13 (1).
(33) Gourlot, C.; Gosset, A.; Glattard, E.; Aisenbrey, C.; Rangasamy, S.; Rabineau, M.; Ouk, T. S.; Sol, V.; Lavalle, P.; Gourlaouen, C.; Ventura, B.; Bechinger, B.; Heitz, V. Antibacterial Photodynamic Therapy in the Near-Infrared Region with a Targeting Antimicrobial Peptide Connected to a T-Extended Porphyrin. ACS Infect Dis 2022, 8 (8), 1509-1520.
(34) Guo, F. C.; Chen, C. Y. Zwitterionic Core-Sheath Nanofibers in Antibacterial Photodynamic Therapy. ACS Appl Polym Mater 2022, 4 (6), 4576-4587.
(35) Scheberl, A.; Khalil, M. L.; Maghsoodi, F.; Strach, E. W.; Yang, J.; Chi, E. Y.; Schanze, K. S.; Reimhult, E.; Whitten, D. G. Quantitative Determination of Dark and Light-Activated Antimicrobial Activity of Poly(Phenylene Ethynylene), Polythiophene, and Oligo (Phenylene Ethynylene) Electrolytes. ACS Appl Mater Interfaces 2020, 12 (19), 21322-21329.

1. A compound having the chemical structure selected form:

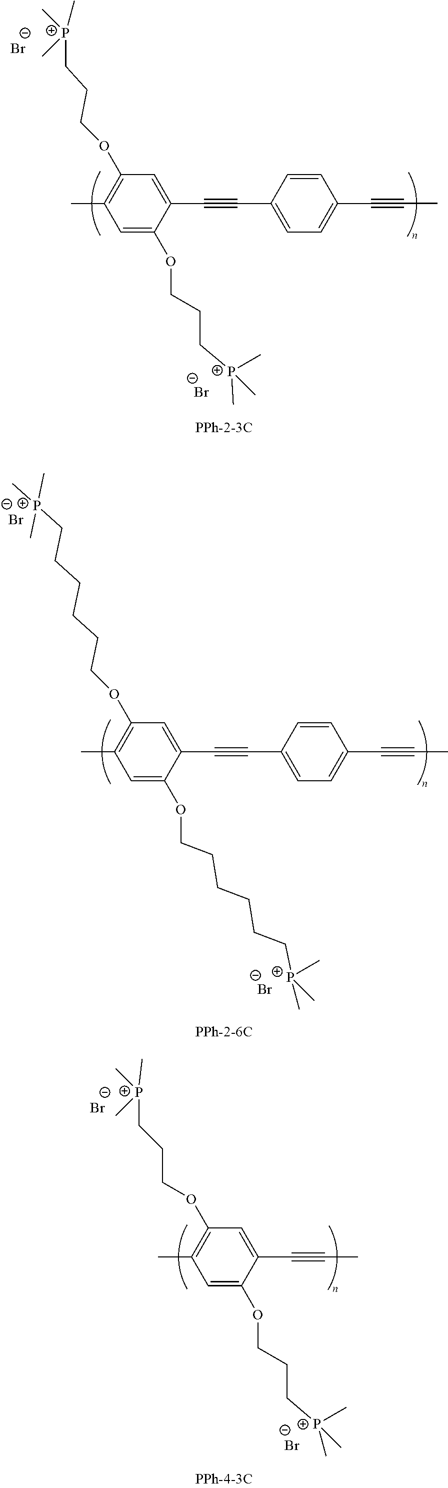

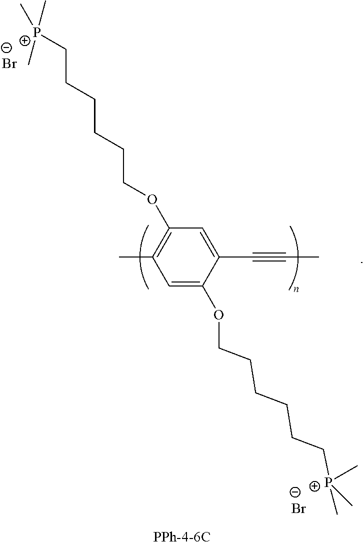

2. An antibacterial composition comprising a compound of claim 1 and pharmaceutically acceptable carrier.

3. A method of treating a condition, the method comprising administering to a subject in need thereof a therapeutically-effective amount of a compound of claim 1 or an antibacterial composition of claim 2, and irradiating the compound.

4. The method of claim 3, wherein the compound is PPh-2-6C, PPh-4-6C, or a pharmaceutically-acceptable salt thereof.

5. The method of claim 3, wherein the compound is irradiated with light having a wavelength of about 200 nm to about 800 nm.

6. The method of claim 3, wherein the condition is an infection.

7. The method of claim 6, wherein the infection is caused by a microbe.

8. The method of claim 7, wherein the microbe is a bacterium.

9. The method of claim 7, wherein the microbe is a Gram-positive bacterium.

10. The method of claim 7, wherein the microbe is a Gram-negative bacterium.

11. The method of claim 7, wherein the microbe is a drug resistant bacterium.

12. The method of claim 7, wherein the microbe is methicillin-resistant Staphylococcus aureus.

13. The method of claim 7, wherein the microbe is Acinetobacter baumannii.
